(19) United States
(12) Patent Application Publication
(10) Pub. No.: US 2021/0311286 A1
(43) Pub. Date: Oct. 7, 2021

(54) OPTHALMIC IMAGING SYSTEM AND OPTICAL IMAGING APPARATUS INCLUDING THE SAME

(71) Applicant: INTHESMART Co., Ltd., Seoul (KR)

(72) Inventors: Choong Hee LEE, Seoul (KR); Uk KANG, Seoul (KR); Il Hyung SHIN, Jeju (KR); Volkov Dmitrii Yurievich, Saint-Petersburg (RU); Papayan Garri Vazgenovich, Saint-Petersburg (RU)

(73) Assignee: INTHESMART Co., Ltd., Seoul (KR)

(21) Appl. No.: 16/937,597

(22) Filed: Jul. 24, 2020

(30) Foreign Application Priority Data
Apr. 1, 2020 (KR) 10-2020-0039657

(51) Int. Cl. G02B 9/06; G02B 7/04; G02B 27/14; A61B 3/12; A61B 3/14; A61B 3/00

(52) U.S. Cl. CPC G02B 9/06; G02B 7/04; G02B 27/14; G02B 27/0025; A61B 3/14; A61B 3/0008; A61B 3/12

(57) ABSTRACT

An ophthalmic imaging system comprises a photographing optical system including an ophthalmic lens module including a first positive lens and a first converging lens sequentially arranged from the examinee's fundus and a projection lens module including a negative meniscus with a convex surface facing in an opposite direction of the examinee's fundus, a second positive lens, a diverging lens, and a second converging lens sequentially arranged from the examinee's fundus wherein the following conditional expression is satisfied, S′p/Sp≥2.8, Sp≥30 mm, wherein Sp, Sp′ is a first distance from a paraxial plane of the ophthalmic lens module to an entrance pupil plane and a second distance from a paraxial plane of the ophthalmic lens module to an exit pupil plane.

### BACKGROUND OF THE INVENTION

### Technical Field

The present invention relates to an ophthalmic imaging system capable of visualizing a fundus and an ophthalmic imaging apparatus including the same.

### Description of the Related Art

In general, an optical system for imaging the fundus consists of a special ophthalmic lens, a light projection lens, and an aperture stop disposed therebetween.

In particular, an exit pupil position of the ophthalmic lens should be in an image space with a predetermined distance (usually, a distance less than 3-4 times the focal length of the ophthalmic lens) in order to optically connect the ophthalmic lens, the optical projection lens disposed rearward of the ophthalmic lens and the optical illumination lens disposed laterally.

The optical system should improve an optical resolution to account for the fundus image while securing a sufficient viewing angle. It also needs improved optical properties such as a chromatic aberration correction by appropriately combining a shape of lens and lenses included in imaging optics.

### SUMMARY OF DISCLOSURE

In embodiments, an ophthalmic imaging system comprises: an illumination optical system that illuminates an examinee's fundus with light emitted from a light source; and a photographing optical system that forms an optical path of the light reflected from the examinee's fundus; wherein the photographing optical system comprises, an ophthalmic lens module including a first positive lens and a first converging lens sequentially arranged from the examinee's fundus, a projection lens module including a negative meniscus with a convex surface facing toward the examinee's fundus, a second positive lens, a diverging lens, and a second converging lens sequentially arranged from the examinee's fundus, and an aperture stop disposed on an optical axis of the light reflected from the examinee's fundus between the ophthalmic lens module and the projection lens module, wherein the following conditional expression is satisfied: S′p/Sp≥2.8, Sp≥30 mm, wherein Sp, Sp′ is a first distance from a paraxial plane of the ophthalmic lens module to an entrance pupil plane and a second distance from a paraxial plane of the ophthalmic lens module to an exit pupil plane.

The first positive lens may be formed in a convex form on both sides as a single lens or formed as a positive meniscus in which a convex surface thereof faces in an opposite direction of the examinee's fundus.

The first converging lens may be formed by bonding a main positive lens and a first negative meniscus having a convex surface facing in the opposite direction of the examinee's fundus.

The second positive lens may be formed as a positive meniscus in which a convex surface thereof faces toward the examinee's fundus or formed in a convex form on both sides, or formed in a positive meniscus in which a convex surface thereof faces in an opposite direction of the examinee's fundus.

The diverging lens may be formed by bonding a first negative lens and a convex lens on both sides.

The first negative lens may be formed as at least one of a lens having a concave form on both sides, a plano-concave, a negative meniscus.

The second converging lens may be formed by bonding a biconvex lens and a negative lens.

The ophthalmic imaging system may further comprises a mounter disposed on a side of the second converging lens so as to be movable along the optical axis and a driving motor for driving the mounter.

The ophthalmic imaging system satisfies the following conditional expressions, n1=(1.0, . . . , 1.5)n62, n21=(0.95, . . . , 1.05)n61, n22=(0.95, . . . , 1.05)n51, n3=(0.8, . . . , 1.1)n4, n52∈[1.4, . . . , 1.5], wherein ni is a refractive index of the i-th lens from the examinee's fundus toward an image receiving unit and nij is the refractive index of the j-th lens bonded to the i-th lens.

The ophthalmic imaging system satisfies the following conditional expressions ν1∈[25, . . . , 50], ν21=(1.3, . . . , 2.2)ν22, ν3∈[17, . . . , 30]=(1.0, . . . , 1.6)ν4, ν51∈[25, . . . , 35]=(0.65, . . . , 0.75)ν62, ν61∈[65, . . . , 70]=(1.45, . . . , 1.8)ν52, wherein νi is an Abbe number of a material of the i-th lens from the examinee's fundus toward an image receiving unit and νij is the Abbe number of the material of the j-th lens bonded to the i-th lens.

The ophthalmic imaging system satisfies the following conditional expression, 1.1≤f′p/f′o≤1.3, wherein f′o and f′p are focal lengths of the ophthalmic lens module and the projection lens module respectively.

A chief ray may be close to parallel to an optical axis of the projection lens module from the paraxial plane of the projection lens module to the image receiving unit.

In embodiments, an ophthalmic imaging apparatus comprises: an imaging unit for photographing an examinee's fundus; and an image generator for generating a fundus image by processing an image of the examinee's fundus photographed by the imaging unit wherein the imaging unit comprises, an illumination optical system that illuminates an examinee's fundus with light emitted from a light source; a photographing optical system that forms an optical path of the light reflected from the examinee's fundus; an image receiving unit disposed spaced apart from the photographing optical system with a predetermined interval; and an optical splitter disposed between the photographing optical system and the image receiving unit. wherein the photographing optical system comprises, an ophthalmic lens module including a first positive lens and a first converging lens sequentially arranged from the examinee's fundus, a projection lens module including a negative meniscus with a convex surface facing toward the examinee's fundus, a second positive lens, a diverging lens, and a second converging lens sequentially arranged from the examinee's fundus, and an aperture stop disposed on an optical axis of the light reflected from the examinee's fundus between the ophthalmic lens module and the projection lens module.

The optical splitter may include a beam splitter for separating an amount of light incident through the optical path from the examinee's fundus and an aiming light source spaced apart from the beam splitter with a predetermined distance, disposed at a perpendicular direction to the optical path.

The aiming light source may include a main light source and an auxiliary light source spaced apart from the main light source.

A wavelength of light emitted from the aiming light source may have a wavelength of infrared light or near infrared light.

An examinee's viewing angle based on the main light source may be symmetric about the optical axis and the examinee's viewing angle based on the auxiliary light source may be asymmetric about the optical axis.

An optical path of a first light emitted from the main light source may be different from that of a second light emitted from the auxiliary light source.

### BRIEF DESCRIPTION OF THE DRAWINGS

References will be made to embodiments of the invention, examples of which may be illustrated in the accompanying figures. These figures are intended to be illustrative, not limiting. Although the invention is generally described in the context of these embodiments, it should be understood that it is not intended to limit the scope of the invention to these particular embodiments.

FIG. 1 shows a schematic diagram of an ophthalmic imaging apparatus according to embodiments of the present invention.

FIG. 2 shows an electronic block diagram of an ophthalmic imaging apparatus according to embodiments of the present invention

FIG. 3 is a schematic diagram of an optical imaging system for an ophthalmic imaging apparatus according to embodiments of the present invention.

FIG. 4 is a schematic diagram of an ophthalmic lens module of the photographing optical system according to embodiments of the present invention.

FIG. 5 is a schematic diagram of a projection lens module of the photographing optical system according to embodiments of the present invention.

FIG. 6 illustrates an optical characteristic based on a structure of ophthalmic lens module of an ophthalmic imaging system according to embodiments of the present invention.

FIG. 7 is a diagram showing an optical path of a chief rays based on an arrangement of an optical system in an optical imaging system according to embodiments of the present invention.

FIGS. 8A and 8B are diagrams for explaining the operation of the ophthalmic imaging system according to embodiments of the present invention.

### DETAILED DESCRIPTION OF THE PREFERRED EMBODIMENTS

In the following description, for purposes of explanation, specific details are set forth in order to provide an understanding of the invention. It will be apparent, however, to one skilled in the art that the invention can be practiced without these details. Furthermore, one skilled in the art will recognize that embodiments of the present invention, described below, may be implemented in a variety of ways, such as a process, an apparatus, a system, a device, or a method on a tangible computer-readable medium.

Components shown in diagrams are illustrative of exemplary embodiments of the invention and are meant to avoid obscuring the invention. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components that may be implemented in software, hardware, or a combination thereof.

It shall also be noted that the terms “coupled” “connected” or “communicatively coupled” shall be understood to include direct connections, indirect connections through one or more intermediary devices, and wireless connections.

Furthermore, one skilled in the art shall recognize: (1) that certain steps may optionally be performed; (2) that steps may not be limited to the specific order set forth herein; and (3) that certain steps may be performed in different orders, including being done contemporaneously.

Reference in the specification to “one embodiment,” “preferred embodiment,” “an embodiment,” or “embodiments” means that a particular feature, structure, characteristic, or function described in connection with the embodiment is included in at least one embodiment of the invention and may be in more than one embodiment. The appearances of the phrases “in one embodiment,” “in an embodiment,” or “in embodiments” in various places in the specification are not necessarily all referring to the same embodiment or embodiments.

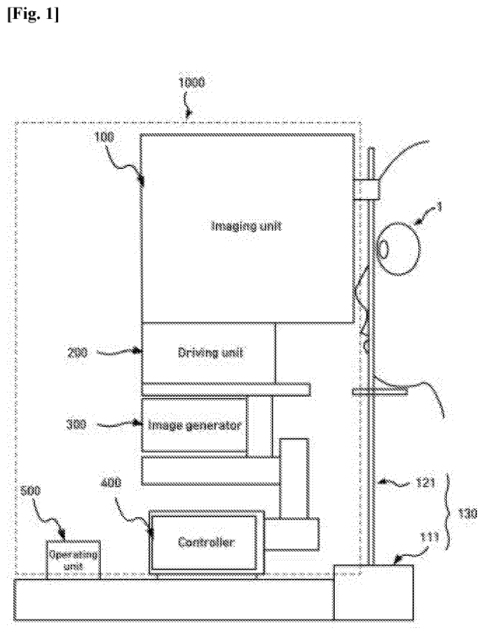

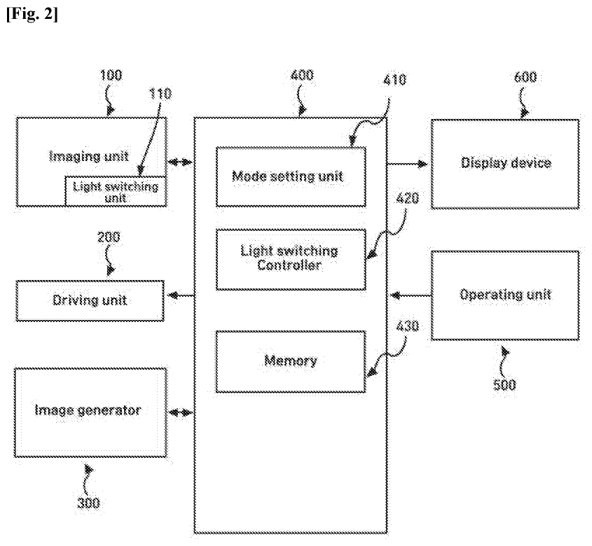

FIG. 1 shows a schematic diagram of an ophthalmic imaging apparatus according to embodiments of the present invention and FIG. 2 shows an electronic block diagram of an ophthalmic imaging apparatus according to embodiments of the present invention. As depicted in FIGS. 1 and 2, the ophthalmic imaging apparatus 1000 may include an imaging unit 100, a driving unit 200, an image generator 300, a controller 400, an operating unit 500 and a display device 600. Also, as shown in FIG. 1, the ophthalmic imaging apparatus may include a support 130 having a base plate 111 and a head support 121, and may acquire an image of the fundus of a subject(examinee) supported by the head support 121. The support 130 may be configured in various forms, a detailed description thereof is omitted since the various forms may be easily implemented by those skilled in the art.

In embodiments, the imaging unit 100 includes an illumination lens module constituting the illumination optical system and a photographing lens module constituting the photographing optical system, for example, an ophthalmic lens module, a projection lens module, and the like. The illumination lens module may include a visible light source and an infrared light source, and a light switching unit 110 in which selectively switches the visible light source and the infrared light source so that light emitted from the visible light source or the infrared light source illuminates on the subject's fundus. The light switching unit 110 may be a mechanical unit such as a beam splitter, and may be replaced with a process of an electronic signal. The light switching unit 110 may be selectively operated under a control of a controller 400. More detailed configurations and operation methods of the imaging unit 100 is given below.

In embodiments, the driving unit 200 may selectively drive internal components of the imaging unit 100, for example, an illumination lens module, in response to the selected light source under the control of the controller 400. In addition, the driving unit 200 may include a motor driving unit for moving a mounter.

In embodiments, the image generator 300 generates a fundus image by processing the fundus area photographed by the imaging unit 100 under the control of the controller 400, and outputs the fundus image. Also, the image generator 300 saves the fundus image in a memory 430 or display on a display device 600.

In embodiments, the operating unit 500 includes a variety of manipulation means for selecting mode selection means and lens focus operation means that a medical staff such as an ophthalmologist and an ophthalmology nurse can select a visible light imaging mode and an infrared light imaging mode according to embodiments of the present invention. A signal (command) generated through selection means and operation means is output to the controller 400.

The manipulation means may include at least one or more of a button, a joystick, a touch pad, a mouse, and the like, but is not limited thereto.

In embodiments, the display device 600 displays an operation information according to the operation of the ophthalmic imaging apparatus under the control of the controller 400, and displays at least one of an infrared fundus image and a visible light fundus image according to a mode information the present invention.

In embodiments, the controller 400 may be a CPU, an application processor (AP), a microcontroller, or the like, and includes a mode setting unit 410, a light switching controller 420, and a memory 430 like storage unit. The controller 400 controls overall operation of the ophthalmic imaging apparatus according to the present invention.

In detail, if a mode selection occurs by inputting a mode selection signal by the operation unit 500, the mode setting unit 410 determines whether the mode selection signal is a visible light photographing mode or an infrared photographing mode, and selectively drive internal components of the imaging unit 100 by controlling the driving unit 200 in response to a determined mode.

When a mode is set in the mode setting unit 410, the light switching controller 420 allows the light switching unit 110 to illuminate visible light or infrared light to a examinee's fundus in response to the set mode.

In embodiments, the memory 430 may include a computer-readable medium in the form of volatile memory such as random access memory (RAM), non-volatile memory such as read only memory (ROM) and flash memory, and the like. The memory may 430 include, but is not limited to a disk drive such as a hard disk drive, a solid state drive, an optical disk drive, and the like. In addition, the memory 430 may include a program area for storing a control program in order to control an overall operation of the ophthalmic imaging apparatus according to the present invention, a temporary area for temporarily storing data generated during controlling the control program, and a data area for storing information and images inputted through the operation unit 500.

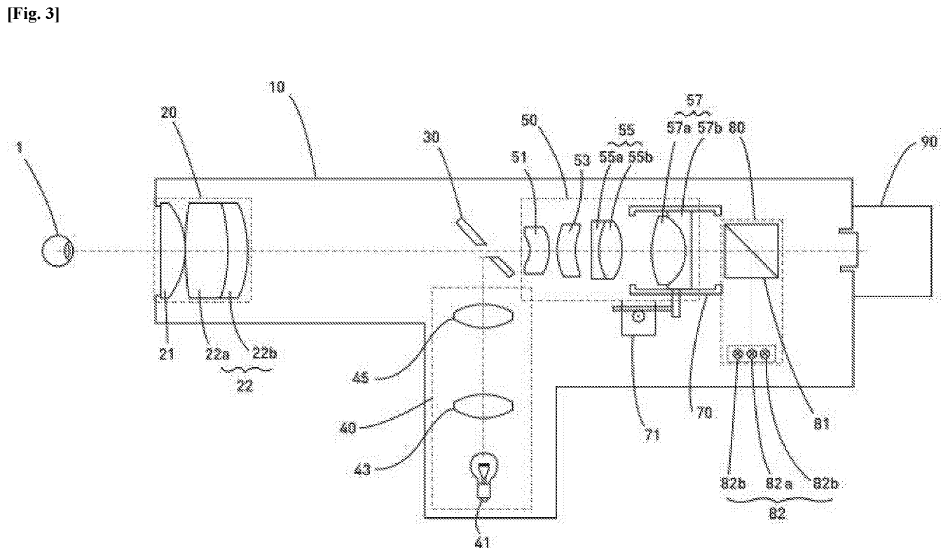

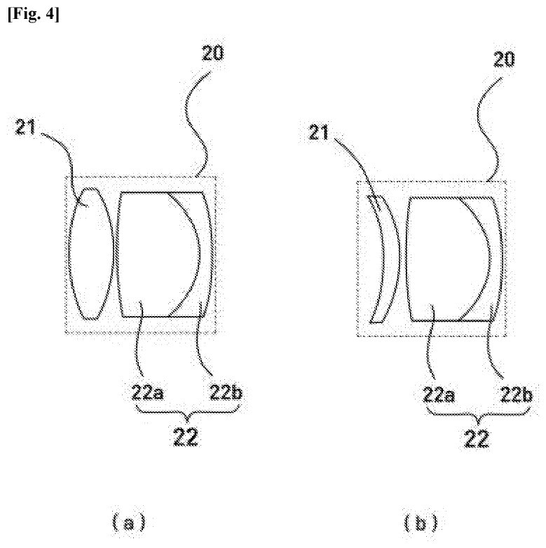

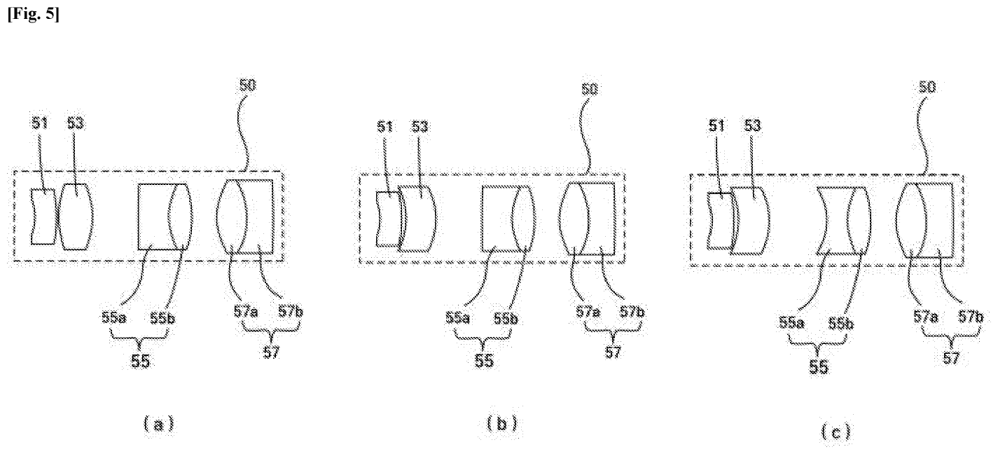

FIG. 3 is a schematic diagram of an optical imaging system for an ophthalmic imaging apparatus according to embodiments of the present invention, FIG. 4 is a schematic diagram of an ophthalmic lens module of the photographing optical system according to embodiments of the present invention, and FIG. 5 is a schematic diagram of a projection lens module of the photographing optical system according to embodiments of the present invention.

Referring to FIGS. 3 to 5, the ophthalmic imaging system 10 according to embodiments of the present invention may be inserted into the imaging unit 100 of FIG. 1 described above. The ophthalmic imaging system 10 may include a photographing optical system 20, 30, 50 an illumination optical system 40, an optical splitter 80 and an image receiving unit 90.

In embodiments, the photographing optical system 20, 30, 50 may form an optical path so that light reflected from the examinee's fundus 1 by light irradiated from the illumination optical system 40 is incident on the image receiving unit 90, thereby generating an optical fundus image. The photographing optical system 20, 30, 50 includes an ophthalmic lens module 20 disposed in a direction of the fundus of the examinee, a projection lens module 50 spaced apart from the ophthalmic lens module 20 with a predetermined interval, and aperture stop 30 disposed between the ophthalmic lens module 20 and the projection lens module 50.

In embodiments, the ophthalmic lens module 20 may include a first positive lens 21 and a first converging lens 22 sequentially arranged from the examinee's fundus 1 so that the light reflected from the fundus may converge to a position of the aperture stop 30.

In embodiments, the first positive lens 21 may be a single positive lens. As shown in (a) of FIG. 4, the first positive lens 21 may be formed in a convex form on both sides, as shown in (b) of FIG. 4. The first positive lens 21 may be formed as a positive meniscus having a convex surface facing in an opposite direction of the examinee's fundus 1. In addition, the first converging lens 22 may be formed by bonding a main positive lens 22a and a first negative meniscus 22b having a convex surface facing in the opposite direction of the examinee's fundus 1.

In embodiments, the aperture stop 30 may be an inclined mirror having a hole, and may be disposed on the optical axis of the light reflected from the examinee's fundus 1 between the ophthalmic lens module 20 and the projection lens module 50. The aperture stop 30 combined with a tilted mirror that reflects the light irradiated from a light source 41 of the illumination optical system 40 toward the examinee's fundus(1). In addition, the hole of the aperture stop 30 serves as an optical channel so that the light reflected from the fundus converges through the ophthalmic lens module 20 and proceeds to the projection lens module 50.

The dotted line shown means an optical axis of the light emitted from the illumination optical system 40 and the light reflected from the examinee's fundus.

In embodiments, the projection lens module 50 may be form an optical path so that the light passing through the aperture stop 30 is incident on the image receiving unit 90. The projection lens module 50 may include a negative meniscus 51 a second positive lens 53, a diverging lens 55, and a second converging lens 57 that is sequentially arranged from the examinee's fundus 1.

In embodiments, the negative meniscus 51 may be formed such that a convex surface thereof faces toward in a opposite direction of the examinee's fundus.

In embodiments, the second positive lens 53 may be formed as a positive meniscus in which a convex surface thereof faces toward the examinee's fundus

In embodiments, as illustrated in (a) of FIG. 5, the second positive lens 53 may be formed in a convex form on both sides. In addition, as illustrated in (b) and (c) of FIG. 5, the second positive lens 53 may be formed in a positive meniscus facing in an opposite direction of the examinee's fundus 1.

In embodiments, the diverging lens 55 may be formed by bonding a first negative lens 55a and a convex lens on both sides 55b. In this case, as illustrated in (a), (b), (c) of FIG. 5, the first negative lens 55a may be formed as a plano-concave, may be a negative meniscus and may be formed as a lens having a concave form on both sides, respectively.

In embodiments, the second converging lens 57 may be formed by bonding a biconvex lens 57a and a negative lens 57b.

As described above, the first positive lens 21 and the first converging lens 22 constituting the ophthalmic lens module 20 and the negative meniscus 51, the second positive lens 53, the diverging lens 55, and the second converging lens 57 constituting the projection lens module 50, have the following conditional expressions.

n₁=(1.0 . . . 1.5)n₆₂

n₂₁=(0.95 . . . 1.05)n₆₁

n₂₂=(0.95 . . . 1.05)n₅₁

n₃=(0.8 . . . 1.1)n₄

n₅₂∈[1.4 . . . 1.5]

Here, ni is a refractive index of the i-th lens from the examinee's fundus 1 toward the image receiving unit 90, and nij is the refractive index of the j-th lens bonded to the i-th lens. For example, n₆₂ means the refractive index of the second lens(concave lens 57) of the sixth lens(second converging lens 57) from the examinee's fundus, and n₂₁ means the refractive index of the first lens(main positive lens 22a) of the second lens(first converging lens 22) from the examinee's fundus.

Also the first positive lens 21 and the first converging lens 22 constituting the ophthalmic lens module 20 and the negative meniscus 51, the second positive lens 53, the diverging lens 55, and the second converging lens 57 constituting the projection lens module 50, satisfies the following conditional expressions.

ν₁∈[25 . . . 50]

ν₂₁=(1.3 . . . 2.2)ν₂₂

ν₃∈[17 . . . 30]=(1.0 . . . 1.6)ν₄

ν₅₁∈[25 . . . 35]=(0.65 . . . 0.75)ν₆₂

ν₆₁∈[65 . . . 70]=(1.45 . . . 1.8)ν₅₂

Here, νi is an Abbe number of a material of the i-th lens from the examinee's fundus 1 toward the image receiving unit 90, and νij is the Abbe number of the material of the j-th lens bonded to the i-th lens. For example, ν₆₂ means the Abbe number of the material of the second lens(concave lens 57) of the sixth lens(second converging lens 57) from the examinee's fundus, and ν₂₁ means the Abbe number of the material of the first lens(main positive lens 22a) of the second lens(first converging lens 22) from the examinee's fundus.

Thus, the photographing optical system including lenses in which satisfies the refractive index and the Abbe number almost completely compensates for an increased chromatic aberration in an optical spectrum of the wide wavelength range of 0.49 μm to 0.9 μm while the light reflected from the fundus passes through the entire lenses.

In embodiments, the illumination optical system 40 may be disposed at the side of the optical path of the photographing optical systems 20, 30, 50 to form an optical path for irradiating light emitted from the light source 41 to the examinee's fundus. The illumination optical system 40 may include a light source 41, a first lens group 43, and a second lens group 45. Both lens groups may also include a set of special diaphragms and black dots to prevent ghosts and reflects from the patient's eye as well as the lenses 20 of the photographing optical system. The light source 41 may be a visible light source or a near-infrared light source. Combination of the first lens group 43 and the second lens 45 together forms an optical system 40 that creates an image of a light source 41 near a mirror 30 directing the light into the ophthalmic lens 20, which builds a projection image of the light source 41 on the cornea of the patient's eye. Thus, the examinee's fundus is illuminated uniformly within the working viewing angle of the photographing optical system 20, 30, 50. The first lens group 43 may be formed as a diffusion lens to diffuse light emitted from the light source 41, and the second lens group 45 may be formed as an illumination lens for irradiating the incoming light from the diffusion lens at a predetermined exit angle.

In embodiments, the optical splitter 80 may include a beam splitter 81 for separating an amount of light incident through the optical path from the fundus and an aiming light source 82 spaced apart from the beam splitter 81 with a predetermined distance. The aiming light source 82 may be disposed at a perpendicular direction to the optical path. The beam splitter 81 may include a plate beam splitter, a cube beam splitter, or the like. The aiming light source 82 may be formed of an LED, and may include a main light source 82a and an auxiliary light source 82b spaced apart from the main light source 82a with a predetermined interval. In addition, a wavelength of the light emitted from the aiming light source 82 may have a wavelength range of infrared light or near infrared light.

In embodiments, the image receiving unit 90 may be disposed spaced apart from the optical splitter 80 with a predetermined interval and include an image sensor (not shown). The image sensor converts an input light into a fundus image signal. In this case, the image sensor may be a charge coupled device (CCD) image sensor or a complementary metal oxide semiconductor (CMOS) image sensor.

In addition, the ophthalmic imaging system 10 may include a mounter 70 in which the second converging lens 57 of the projection lens module 50 is capable of moving along the optical axis, and a motor driver 71 linked to the mounter 70. If the examinee's eye is ametropia, a defocus shown in the image receiving unit 90 can be compensated because the second converging lens 57 is movable along the optical axis. For example, in one embodiment of the present invention, a shift value d for compensating 10 diopters has the following conditional expression.

d=(0.025±0.003)f′6

Here, f′6 means a focal length of the sixth lens, the sixth lens is the second converging lens 57 from the examinee's fundus toward the image receiving unit 90 according to one embodiment of the present invention.

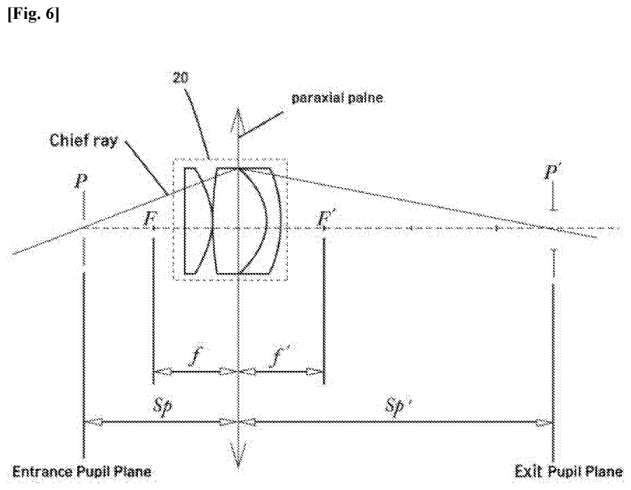

FIG. 6 illustrates an optical characteristic based on a structure of ophthalmic lens module of an ophthalmic imaging system according to embodiments of the present invention.

Referring to FIG. 6, in the ophthalmic lens module 20 according to embodiments of the present invention, F and F′ are a front and back focal positions of the ophthalmic lens module 20, each of P and P′ are an entrance pupil plane and an exit pupil plane of a chief ray, f, f′ is a focal length of the ophthalmic lens module 20, Sp, Sp′ is a first distance from a paraxial plane of the ophthalmic lens module 20 to an entrance pupil plane and a second distance from a paraxial plane of the ophthalmic lens module 20 to an exit pupil plane, respectively. Here, the ophthalmic lens module 20 is arranged to satisfy the following conditional expression S′p/Sp≥2.8, Sp≥30 mm. That is, the ophthalmic imaging system can have a wide viewing angle by allowing the first distance (Sp) to be 30 mm or more and a ratio of the first distance (Sp) to a second distance (Sp′) to be 2.8 or more.

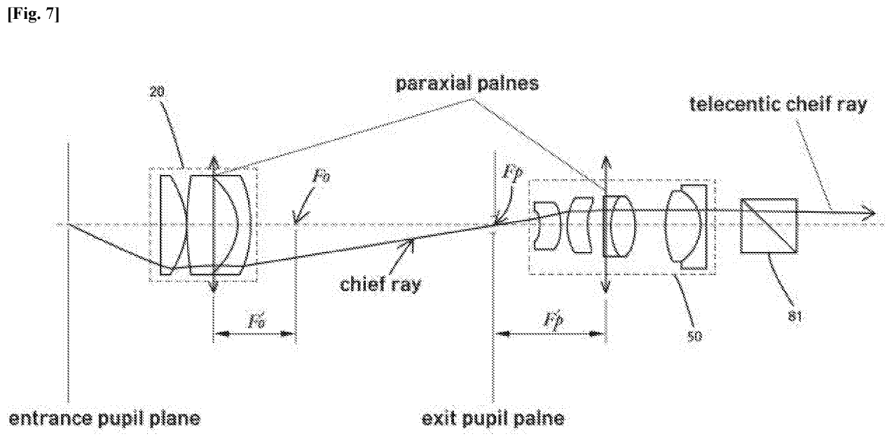

FIG. 7 is a diagram showing an optical path of a chief rays based on an arrangement of an optical system in an optical imaging system according to embodiments of the present invention.

Referring to FIG. 7, in the photographing optical system according to embodiments of the present invention, Fo and Fp are a focal points positions of the ophthalmic lens module 20 and a focal position of the projection lens module 50, respectively, and f′o and f′p are focal lengths of the ophthalmic lens module 20 and the projection lens module 50 respectively.

Of components constituting the photographing optical system, the projection lens module 50 has a same shape, a same direction, and a same position as illustrated in the projection lens module in FIG. 3. Accordingly, the chief ray passes through the entrance pupil plane, passes through the ophthalmic lens module 20, converges to the exit pupil plane located at the front end of the projection lens module 50, and is parallel to the optical axis of the projection lens module 50 from a paraxial plane of the projection lens module 50 to the image receiving unit 90.

In this case, the lens modules included in the photographing optical system are disposed to satisfy the following conditional expression 1.1≤f′p/f′o≤1.3. Accordingly, the photographing optical system can effectively compensate for a residual aberrations caused by the ophthalmic lens module 20 disposed in front of the projection lens module 50.

Based on the above conditions, as a specific experimental example, the following parameters were applied to the ophthalmic imaging system according to an embodiment of the present invention. For example, a first distance (Sp) from a paraxial plane of the ophthalmic lens module 20 to an entrance pupil plane of the chief ray having 31 mm, a viewing angle (a) having 47° in a target space, a diameter (Dp) of the entrance pupil having 1.5 mm, a working spectrum range (Δλ) having 0.49-0.9 microns, a distance(f′o) from the paraxial plane of the ophthalmic lens module 20 to the focal position of the ophthalmic lens module 20 having 30 mm (33 diopters), a diameter (y) of an image at the image receiving unit having 10 mm, a diameter of the aperture stop having 4 mm, a diopter compensation range of ±35 diopters or more, a Strehl ratio of 0.9 or more, and a length (L) of 265 mm from a surface of the first positive lens 21 to an image plane of the image receiving unit. As a result, it is possible to improve the quality of the fundus image without a use of aspherical lens to the ophthalmic imaging system and to increase the viewing angle and a diameter of an entrance pupil.

With regard to polychromatic diffraction wavelength aberration lists shown according to the optical design parameters applied to the ophthalmic imaging system of the present invention and polychromatic Diffraction MTF lists according to a modulation transfer functions (MTFs) under the above conditions, Numerical embodiments 1 to 5 will now be described.

In surface data of the numerical embodiments, a radius(r) represents the radius of curvature of each optical surface, and d represents an on-axis interval (distance along the optical axis) between a m-th surface and a (m+1)-th surface, where m represents a number of the surfaces from the light incident side, Nd represents the refractive index of each optical member at the d-line, vd represents the Abbe number of each optical member at the d-line.

Numerical embodiment 1
1. Surface Data (mm/unit)

| Surface number | r | d | Nd | vd |
|---|---|---|---|---|
| 1 | ∞ | 9.2 | 1.816 | 46.6 |
| 2 | −30.71 | 0.3 | | |
| 3 | 120.5 | 18.6 | 1.595 | 67.7 |
| 4 | −21.2 | 4.8 | 1.917 | 31.6 |
| 5 | −46.13 | 89 | | |
| STOP | ∞ | 18.5 | | |
| 7 | −10.093 | 7.7 | 1.785 | 26.3 |
| 8 | −16.181 | 2.8 | | |
| 9 | 20.42 | 7.2 | 1.959 | 17.5 |
| 10 | 22.39 | 6.2 | | |
| 11 | ∞ | 2.7 | 1.917 | 31.6 |
| 12 | 20.8 | 9 | 1.439 | 94.9 |
| 13 | −20.8 | 11.4 | | |
| 14 | 31.8 | 12.9 | 1.595 | 67.7 |
| 15 | −15.596 | 2.5 | 1.613 | 44.3 |
| 16 | −270.4 | 12.5 | | |
| 17 | ∞ | 20 | Beam splitter cube | |
| 18 | ∞ | 29.4 | | |

2. Wave aberration lists

| | | | | |
|---|---|---|---|---|
| Pupil | 0.500000 | 0.587562 | 0.656273 | 0.800000 |

Entrance field 0 deg

| | | | | |
|---|---|---|---|---|
| −1.000 | 0.012710 | 0.021004 | 0.090240 | 0.131476 |
| −0.800 | 0.020713 | 0.034300 | 0.079453 | 0.104070 |
| −0.600 | 0.017477 | 0.028459 | 0.054133 | 0.067130 |
| −0.400 | 0.009679 | 0.015557 | 0.027035 | 0.032526 |
| 0.000 | 0.000000 | 0.000000 | 0.000000 | 0.000000 |

Tangential fan, entrance field 12 deg

| | | | | |
|---|---|---|---|---|
| −1.000 | −0.155803 | −0.019641 | 0.187094 | 0.613648 |
| −0.800 | −0.069053 | 0.017505 | 0.161426 | 0.482901 |
| −0.600 | −0.026135 | 0.023006 | 0.115380 | 0.342697 |
| −0.400 | −0.008959 | 0.014455 | 0.065993 | 0.208751 |
| 0.000 | 0.000000 | 0.000000 | 0.000000 | 0.000000 |
| 0.400 | 0.030034 | 0.017295 | 0.001291 | −0.106350 |
| 0.600 | 0.069470 | 0.035340 | 0.021194 | −0.126797 |
| 0.800 | 0.140692 | 0.057060 | 0.047412 | −0.132846 |
| 1.000 | 0.271063 | 0.087810 | 0.079754 | −0.127473 |

Sagittal fan, entrance field 12 deg

| | | | | |
|---|---|---|---|---|
| −1.000 | 0.005012 | 0.011790 | 0.094216 | 0.161103 |
| −0.800 | 0.017504 | 0.031212 | 0.085037 | 0.125999 |
| −0.600 | 0.016590 | 0.028091 | 0.058731 | 0.080873 |
| −0.400 | 0.009614 | 0.015859 | 0.029569 | 0.039105 |
| 0.000 | 0.000000 | 0.000000 | 0.000000 | 0.000000 |

Tangential fan, entrance field 24 deg

| | | | | |
|---|---|---|---|---|
| −1.000 | 0.186986 | 0.095792 | 0.209370 | 0.688682 |
| −0.800 | 0.119813 | 0.051753 | 0.123310 | 0.481244 |
| −0.600 | 0.066048 | 0.019483 | 0.057236 | 0.303461 |
| −0.400 | 0.029753 | 0.002823 | 0.016296 | 0.163009 |
| 0.000 | 0.000000 | 0.000000 | 0.000000 | 0.000000 |
| 0.400 | −0.004308 | −0.024626 | 0.021037 | −0.018075 |
| 0.600 | 0.009227 | −0.062826 | 0.032702 | 0.034226 |
| 0.800 | 0.067730 | −0.108450 | 0.057579 | 0.155883 |
| 1.000 | 0.238645 | −0.128453 | 0.130347 | 0.401331 |

Sagittal fan, entrance field 24 deg

| | | | | |
|---|---|---|---|---|
| −1.000 | −0.084323 | −0.123689 | −0.012814 | 0.131150 |
| −0.800 | −0.038126 | −0.052101 | 0.020048 | 0.109717 |
| −0.600 | −0.013616 | −0.016921 | 0.024064 | 0.073310 |
| −0.400 | −0.003374 | −0.003476 | 0.014841 | 0.036325 |
| 0.000 | 0.000000 | 0.000000 | 0.000000 | 0.000000 |

3. Polychromatic Diffraction MTF

| | | |
|---|---|---|
| Spatial Frequency | Tangential | Sagittal |

Entrance field 0 deg

| | | |
|---|---|---|
| 25.000000 | 0.839367 | 0.839367 |
| 50.000000 | 0.678705 | 0.678705 |
| 75.000000 | 0.528723 | 0.528723 |
| 100.000000 | 0.395144 | 0.395144 |
| 125.000000 | 0.272512 | 0.272512 |
| 150.000000 | 0.163096 | 0.163096 |
| 175.000000 | 0.084608 | 0.084608 |
| 200.000000 | 0.034374 | 0.034374 |

Entrance field 12 deg

| | | |
|---|---|---|
| 25.000000 | 0.769360 | 0.834238 |
| 50.000000 | 0.513391 | 0.667723 |
| 75.000000 | 0.330450 | 0.516717 |
| 100.000000 | 0.230399 | 0.385585 |
| 125.000000 | 0.177343 | 0.265223 |
| 150.000000 | 0.116537 | 0.157448 |
| 175.000000 | 0.059250 | 0.080539 |
| 200.000000 | 0.025297 | 0.032137 |

Entrance field 24 deg

| | | |
|---|---|---|
| 25.000000 | 0.742834 | 0.830523 |
| 50.000000 | 0.488454 | 0.631410 |
| 75.000000 | 0.376298 | 0.511696 |
| 100.000000 | 0.269295 | 0.376857 |
| 125.000000 | 0.177693 | 0.251234 |
| 150.000000 | 0.097107 | 0.143197 |
| 175.000000 | 0.036752 | 0.070138 |
| 200.000000 | 0.012283 | 0.025895 |

Numerical embodiment 2
1. Surface Data (mm/unit)

| Surface number | r | d | Nd | vd |
|---|---|---|---|---|
| 1 | 395 | 9 | 1.847 | 23.8 |
| 2 | −34.724 | 1.7 | | |
| 3 | 120.451 | 21.7 | 1.603 | 60.6 |
| 4 | −21.033 | 4.3 | 1.855 | 24.8 |
| 5 | −49.77 | 89 | | |
| STOP | ∞ | 18.5 | | |
| 7 | −10.489 | 9 | 1.855 | 24.8 |
| 8 | −17 | 5 | | |
| 9 | 25.58 | 10.5 | 1.959 | 17.5 |
| 10 | 35.1 | 9.3 | | |
| 11 | 295.12 | 8.5 | 1.855 | 24.8 |
| 12 | 17.37 | 8.6 | 1.439 | 94.9 |
| 13 | −34.724 | 10 | | |
| 14 | 30.947 | 13.8 | 1.595 | 67.7 |
| 15 | −15.8 | 2.5 | 1.603 | 38.0 |
| 16 | −153.39 | 12.5 | | |
| 17 | ∞ | 20 | Beam splitter cube | |
| 18 | ∞ | 25 | | |

2. Wave aberration lists

| | | | | |
|---|---|---|---|---|
| Pupil | 0.500000 | 0.587562 | 0.656273 | 0.800000 |

Entrance field 0 deg

| | | | | |
|---|---|---|---|---|
| −1.000 | 0.014226 | 0.027725 | 0.076680 | 0.087046 |
| −0.800 | 0.021285 | 0.039595 | 0.072528 | 0.078158 |
| −0.600 | 0.017726 | 0.031975 | 0.051099 | 0.053735 |
| −0.400 | 0.009793 | 0.017316 | 0.025983 | 0.026967 |
| 0.000 | 0.000000 | 0.000000 | 0.000000 | 0.000000 |

Tangential fan, entrance field 12 deg

| | | | | |
|---|---|---|---|---|
| −1.000 | −0.116414 | −0.044675 | 0.155833 | 0.590136 |
| −0.800 | −0.040516 | −0.005065 | 0.137399 | 0.470280 |
| −0.600 | −0.004693 | 0.006834 | 0.100798 | 0.340421 |
| −0.400 | 0.006649 | 0.005861 | 0.060344 | 0.213788 |
| 0.000 | 0.000000 | 0.000000 | 0.000000 | 0.000000 |
| 0.400 | 0.004538 | 0.005577 | −0.023612 | −0.153786 |
| 0.600 | 0.030358 | 0.009244 | −0.030258 | −0.218183 |
| 0.800 | 0.091769 | 0.012814 | −0.039377 | −0.282505 |
| 1.000 | 0.220689 | 0.024332 | −0.049568 | −0.349434 |

Sagittal fan, entrance field 24 deg

| | | | | |
|---|---|---|---|---|
| −1.000 | 0.030289 | 0.021144 | 0.074649 | 0.102248 |
| −0.800 | 0.031243 | 0.036396 | 0.072640 | 0.089512 |
| −0.600 | 0.023333 | 0.030742 | 0.051894 | 0.060931 |
| −0.400 | 0.012319 | 0.016976 | 0.026594 | 0.030443 |
| 0.000 | 0.000000 | 0.000000 | 0.000000 | 0.000000 |

Tangential fan, entrance field 24 deg

| | | | | |
|---|---|---|---|---|
| −1.000 | 0.072916 | −0.007947 | 0.238739 | 0.960026 |
| −0.800 | 0.040429 | −0.011221 | 0.169869 | 0.722598 |
| −0.600 | 0.016874 | −0.012380 | 0.108908 | 0.500729 |
| −0.400 | 0.003878 | −0.009057 | 0.060297 | 0.302597 |
| 0.000 | 0.000000 | 0.000000 | 0.000000 | 0.000000 |
| 0.400 | 0.001286 | −0.025658 | −0.021625 | −0.125464 |
| 0.600 | −0.000575 | −0.067690 | −0.017383 | −0.079046 |
| 0.800 | −0.005911 | −0.146304 | −0.005908 | 0.080307 |
| 1.000 | −0.030478 | −0.308374 | −0.022764 | 0.370955 |

Sagittal fan, entrance field 24 deg

| | | | | |
|---|---|---|---|---|
| −1.000 | 0.019665 | −0.070118 | 0.005402 | 0.110440 |
| −0.800 | 0.022568 | −0.022064 | 0.028271 | 0.093899 |
| −0.600 | 0.017952 | −0.001869 | 0.027213 | 0.063335 |
| −0.400 | 0.009841 | 0.002636 | 0.015781 | 0.031562 |
| 0.000 | 0.000000 | 0.000000 | 0.000000 | 0.000000 |

3. Polychromatic Diffraction MTF lists

| | | |
|---|---|---|
| Spatial Frequency | Tangential | Sagittal |

Entrance field 0 deg

| | | |
|---|---|---|
| 25.000000 | 0.846448 | 0.846448 |
| 50.000000 | 0.693223 | 0.693223 |
| 75.000000 | 0.546342 | 0.546342 |
| 100.000000 | 0.412693 | 0.412693 |
| 125.000000 | 0.289677 | 0.289677 |
| 150.000000 | 0.179834 | 0.179834 |
| 175.000000 | 0.096747 | 0.096747 |
| 200.000000 | 0.041116 | 0.041116 |

Entrance field 12 deg

| | | |
|---|---|---|
| 25.000000 | 0.774726 | 0.843651 |
| 50.000000 | 0.508420 | 0.686840 |
| 75.000000 | 0.316180 | 0.538605 |
| 100.000000 | 0.240725 | 0.405683 |
| 125.000000 | 0.204332 | 0.283840 |
| 150.000000 | 0.136307 | 0.175219 |
| 175.000000 | 0.071435 | 0.093605 |
| 200.000000 | 0.033699 | 0.039339 |

Entrance field 24 deg

| | | |
|---|---|---|
| 25.000000 | 0.699852 | 0.839286 |
| 50.000000 | 0.467498 | 0.680039 |
| 75.000000 | 0.369704 | 0.532690 |
| 100.000000 | 0.283979 | 0.400369 |
| 125.000000 | 0.191105 | 0.277053 |
| 150.000000 | 0.118629 | 0.167290 |
| 175.000000 | 0.058935 | 0.087294 |
| 200.000000 | 0.021146 | 0.035504 |

Numerical embodiment 3
1. Surface Data (mm/unit)

| Surface number | r | d | Nd | vd |
|---|---|---|---|---|
| 1 | −255 | 8.8 | 1.847 | 23.8 |
| 2 | −30 | 2 | | |
| 3 | 127.1 | 21.7 | 1.652 | 58.6 |
| 4 | −20.972 | 4.8 | 1.855 | 24.8 |
| 5 | −50.96 | 89 | | |
| STOP | ∞ | 18.5 | | |
| 7 | −10.47 | 8.1 | 1.959 | 17.5 |
| 8 | −16.516 | 5 | | |
| 9 | 24.82 | 11 | 1.959 | 17.5 |
| 10 | 31.9 | 5.7 | | |
| 11 | 1567 | 9 | 1.855 | 24.8 |
| 12 | 18.2 | 8.9 | 1.439 | 94.9 |
| 13 | −28.5 | 10 | | |
| 14 | 33.783 | 15 | 1.595 | 67.7 |
| 15 | −16.516 | 3.3 | 1.603 | 38.0 |
| 16 | −107.46 | 11 | | |
| 17 | ∞ | 20 | Beam splitter cube | |
| 18 | ∞ | 25.45 | | |

2. Wave aberration lists

| | | | | |
|---|---|---|---|---|
| Pupil | 0.500000 | 0.587562 | 0.656273 | 0.800000 |

Entrance field 0 deg

| | | | | |
|---|---|---|---|---|
| −1.000 | −0.043086 | −0.013078 | 0.037495 | 0.048875 |
| −0.800 | −0.026914 | −0.000670 | 0.033078 | 0.040335 |
| −0.600 | −0.014806 | 0.002877 | 0.022415 | 0.026457 |
| −0.400 | −0.006471 | 0.002279 | 0.011129 | 0.012907 |
| 0.000 | 0.000000 | 000000 | 0.000000 | 0.000000 |

Tangential fan, entrance field 12 deg

| | | | | |
|---|---|---|---|---|
| −1.000 | −0.131218 | −0.004015 | 0.204305 | 0.643463 |
| −0.800 | −0.071893 | 0.011986 | 0.163736 | 0.504583 |
| −0.600 | −0.036967 | 0.013131 | 0.115893 | 0.363875 |
| −0.400 | −0.017929 | 0.007868 | 0.069176 | 0.229464 |
| 0.000 | 0.000000 | 0.000000 | 0.000000 | 0.000000 |
| 0.400 | 0.027487 | 0.008270 | −0.028824 | −0.169346 |
| 0.600 | 0.058663 | 0.015581 | −0.034643 | −0.238790 |
| 0.800 | 0.113707 | 0.022101 | −0.041406 | −0.306291 |
| 1.000 | 0.211211 | 0.028997 | −0.052729 | −0.378242 |

Sagittal fan, entrance field 12 deg

| | | | | |
|---|---|---|---|---|
| −1.000 | −0.000063 | 0.015208 | 0.070553 | 0.095711 |
| −0.800 | 0.003086 | 0.020836 | 0.057834 | 0.073813 |
| −0.600 | 0.003257 | 0.016554 | 0.037998 | 0.046889 |
| −0.400 | 0.001959 | 0.008880 | 0.018599 | 0.022511 |
| 0.000 | 0.000000 | 0.000000 | 0.000000 | 0.000000 |

Tangential fan, entrance field 24 deg

| | | | | |
|---|---|---|---|---|
| −1.000 | 0.255661 | 0.022334 | 0.110315 | 0.605896 |
| −0.800 | 0.167762 | −0.002264 | 0.057977 | 0.439893 |
| −0.600 | 0.101450 | −0.012236 | 0.024253 | 0.297002 |
| −0.400 | 0.054193 | −0.010754 | 0.007108 | 0.177755 |
| 0.000 | 0.000000 | 0.000000 | 0.000000 | 0.000000 |
| 0.400 | −0.012148 | −0.026553 | −0.013726 | −0.114808 |
| 0.600 | 0.018870 | −0.056722 | −0.025076 | −0.139038 |
| 0.800 | 0.117134 | −0.077693 | −0.018972 | −0.111857 |
| 1.000 | 0.351766 | −0.047637 | 0.046075 | 0.019240 |

Sagittal fan, entrance field 24 deg

| | | | | |
|---|---|---|---|---|
| −1.000 | 0.014815 | −0.053886 | 0.006872 | 0.077805 |
| −0.800 | 0.014715 | −0.019394 | 0.021368 | 0.066358 |
| −0.600 | 0.011036 | −0.004049 | 0.019629 | 0.044691 |
| −0.400 | 0.005881 | 0.000431 | 0.011176 | 0.022222 |
| 0.000 | 0.000000 | 0.000000 | 0.000000 | 0.000000 |

3. Polychromatic Diffraction MTF lists

| | | |
|---|---|---|
| Spatial Frequency | Tangential | Sagittal |

Entrance field 0 deg

| | | |
|---|---|---|
| 25.000000 | 0.845911 | 0.845911 |
| 50.000000 | 0.694065 | 0.694065 |
| 75.000000 | 0.545642 | 0.545642 |
| 100.000000 | 0.407157 | 0.407157 |
| 125.000000 | 0.280011 | 0.280011 |
| 150.000000 | 0.168894 | 0.168894 |
| 175.000000 | 0.089019 | 0.089019 |
| 200.000000 | 0.037009 | 0.037009 |

Entrance field 12 deg

| | | |
|---|---|---|
| 25.000000 | 0.757597 | 0.842135 |
| 50.000000 | 0.470368 | 0.685455 |
| 75.000000 | 0.288597 | 0.535590 |
| 100.000000 | 0.238848 | 0.398883 |
| 125.000000 | 0.194104 | 0.273817 |
| 150.000000 | 0.111349 | 0.164004 |
| 175.000000 | 0.057482 | 0.085467 |
| 200.000000 | 0.027423 | 0.034984 |

Entrance field 24 deg

| | | |
|---|---|---|
| 25.000000 | 0.762781 | 0.837937 |
| 50.000000 | 0.521541 | 0.677901 |
| 75.000000 | 0.344627 | 0.526495 |
| 100.000000 | 0.235441 | 0.388676 |
| 125.000000 | 0.161924 | 0.261721 |
| 150.000000 | 0.101426 | 0.152015 |
| 175.000000 | 0.042002 | 0.076117 |

As shown in the numerical embodiments above, the ophthalmic imaging system according to embodiments of the present invention has a wave aberration having an average value of about 0.05 to 0.1 at any wavelength of a working spectral range and not exceeding 0.8. The aberration correction was confirmed by the MTF data, and this value is close to the maximum value at the diameter of entrance pupil having 1.5 mm, which is proposed in embodiments of the present invention.

In general, the maximum resolution of the optical imaging system is determined by a diameter of entrance pupil due to a diffraction effect generated by a wave nature of light. In other words, the larger the diameter of the entrance pupil is, the higher the resolution is. However, even in an ideal optical system without an intrinsic aberration, the resolution cannot be infinite. This is why it is limited by the diffraction limit.

However, since the resolution of the optical imaging system according to embodiments of the present invention may have a maximum value close to the diffraction limit determined by the diameter of entrance pupil having 1.5 mm, it means that the aberration occurring in the optical imaging system is completely corrected. In conclusion, the optical imaging system according to embodiments of the present invention has a fundus image with a very high image quality at above proposed conditions without further increasing the diameter of the entrance pupil.

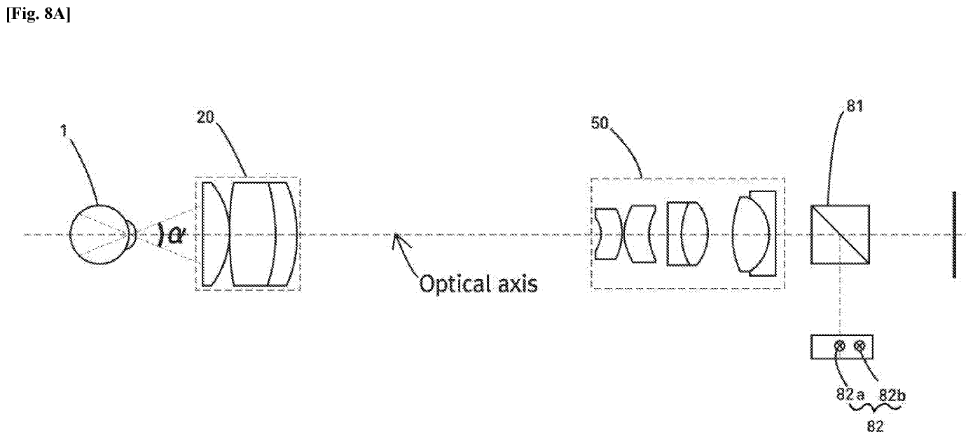

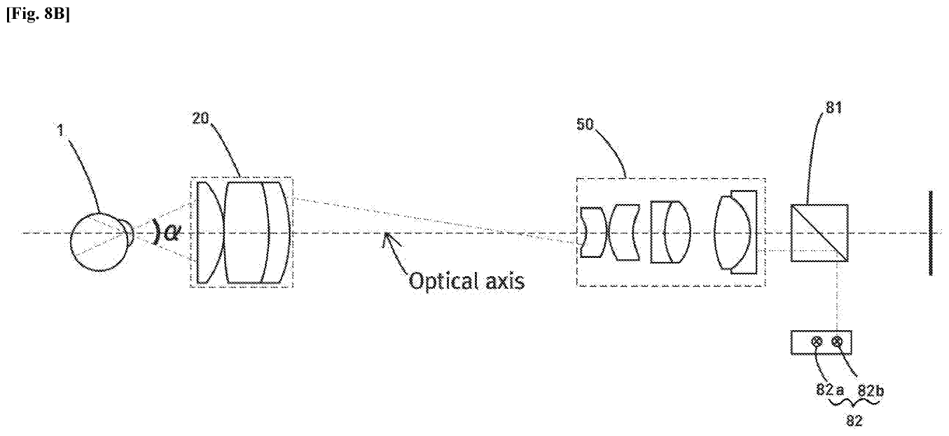

FIGS. 8A and 8B are diagrams for explaining the operation of the ophthalmic imaging system according to embodiments of the present invention.

Referring to FIG. 8A, when the examinee is positioned in front of the ophthalmic imaging system to photograph a fundus image, the main light source 82a is turned on. A first light emitted from the main light source 82a moves along the optical axis of the main light source 82a and is reflected by the beam splitter 81, and then reaches to the examinee's fundus 1 along the optical axis of the photographing optical system 20, 50. The examinee gazes thus at a main light from the main light source 82a, which appears as a green dot. At this time, the photographing optical system 20, 50 projects a portion of the fundus observed on the image receiving unit 90 within the viewing angle (a). The examinee's viewing angle (a) becomes symmetric about the optical axis. Therefore, a primary fundus image which is symmetric about the optical axis can be taken.

As depicted in FIG. 8B, if the auxiliary light source 82b is turned on, the second light emitted from the auxiliary light source 82b moves along the optical axis of the auxiliary light source and is reflected by the beam splitter 81, and then reaches to the examinee's fundus 1 in an optical path different from an optical path of the first light. The examinee thus gazes at the auxiliary light from the auxiliary light source 82b. At this time, the examinee's viewing angle (a) becomes asymmetric about the optical axis of the photographing optical system 20, 50. That is, the examinee does not stare at the optical axis, but looks in a direction different from the optical axis, and thus an additional region of the fundus, which was not visible when examinee viewed the light source 82a, falls within the working angle (a) of the photographic system 20, 50 That is, a secondary fundus image of the examinee may be a fundus region different from fundus region photographed in the primary fundus image.

As described above, the ophthalmic imaging system according to embodiments of the present invention includes an aiming light source that the examinee can stare at a different angle, thereby photographing the fundus image having a wider fundus region portion. Accordingly, it is possible to further secure the information of the lesion obtained through the fundus image, thereby increasing the reliability or accuracy of the fundus image.

It will be appreciated to those skilled in the art that the preceding examples and embodiment are exemplary and not limiting to the scope of the present invention. It is intended that all permutations, enhancements, equivalents, combinations, and improvements thereto that are apparent to those skilled in the art upon a reading of the specification and a study of the drawings are included within the true spirit and scope of the present invention.

What is claimed is:

1. An ophthalmic imaging system comprising:
an illumination optical system that illuminates an examinee's fundus with light emitted from a light source; and
a photographing optical system that forms an optical path of the light reflected from the examinee's fundus;
wherein the photographing optical system comprises,
an ophthalmic lens module including a first positive lens and a first converging lens sequentially arranged from the examinee's fundus,
a projection lens module including a negative meniscus with a convex surface facing in an opposite direction of the examinee's fundus, a second positive lens, a diverging lens, and a second converging lens sequentially arranged from the examinee's fundus, and
an aperture stop disposed on an optical axis of the light reflected from the examinee's fundus between the ophthalmic lens module and the projection lens module,
wherein the following conditional expression is satisfied:
S′p/Sp≥2.8,Sp≥30 mm,
wherein Sp, Sp′ is a first distance from a paraxial plane of the ophthalmic lens module to an entrance pupil plane and a second distance from a paraxial plane of the ophthalmic lens module to an exit pupil plane.

2. The ophthalmic imaging system of claim 1,
wherein the first positive lens is formed as a single biconvex lens or formed as a positive meniscus in which a convex surface thereof faces in an opposite direction of the examinee's fundus.

3. The ophthalmic imaging system of claim 1,
wherein the first converging lens is formed by bonding a main positive lens and a first negative meniscus having a convex surface facing in the opposite direction of the examinee's fundus.

4. The ophthalmic imaging system of claim 1,
wherein the second positive lens is formed as a positive meniscus in which a convex surface thereof faces toward the examinee's fundus or formed in a convex form on both sides, or formed in a positive meniscus in which a convex surface thereof faces in an opposite direction of the examinee's fundus.

5. The ophthalmic imaging system of claim 1,
wherein the diverging lens is formed by bonding a first negative lens and a convex lens on both sides.

6. The ophthalmic imaging system of claim 5,
wherein the first negative lens is formed as at least one of a lens having a concave form on both sides, a plano-concave, a negative meniscus.

7. The ophthalmic imaging system of claim 1,
wherein the second converging lens is formed by bonding a biconvex lens and a negative lens.

8. The ophthalmic imaging system of claim 1, further comprising,
a mounter disposed on a side of the second converging lens so as to be movable along the optical axis and
a driving motor for driving the mounter.

9. The ophthalmic imaging system of claim 1,
wherein the following conditional expressions are satisfied:
n₁=(1.0, . . . ,1.5)n₆₂
n₂₁=(0.95, . . . ,1.05)n₆₁
n₂₂=(0.95, . . . ,1.05)n₅₁
n₃=(0.8, . . . ,1.1)n₄
n₅₂∈[1.4, . . . ,1.5]
wherein ni is a refractive index of the i-th lens from the examinee's fundus toward an image receiving unit and nij is the refractive index of the j-th lens bonded to the i-th lens.

10. The ophthalmic imaging system of claim 1,
wherein the following conditional expressions are satisfied:
ν₁∈[25, . . . ,50]
ν₂₁=(1.3, . . . ,2.2)ν₂₂
ν₃∈[17, . . . ,30]=(1.0, . . . ,1.6)ν₄
ν₅₁∈[25, . . . ,35]=(0.65, . . . ,0.75)ν₆₂
ν₆₁∈[65, . . . ,70]=(1.45, . . . ,1.8)ν₅₂
wherein νi is an Abbe number of a material of the i-th lens from the examinee's fundus toward an image receiving unit and νij is the Abbe number of the material of the j-th lens bonded to the i-th lens.

11. The ophthalmic imaging system of claim 1,
wherein the following conditional expression are satisfied:
1.1≤f′p/f′o≤1.3.
wherein f′o and f′p are focal lengths of the ophthalmic lens module and the projection lens module, respectively.

12. The ophthalmic imaging system of claim 11,
wherein a chief ray is close to parallel to an optical axis of the projection lens module from the paraxial plane of the projection lens module to the image receiving unit.

13. The ophthalmic imaging system of claim 1, further comprising,
an image receiving unit disposed spaced apart from the projection lens module with a predetermined interval and
an optical splitter disposed between the projection lens module and the image receiving unit.

14. The ophthalmic imaging system of claim 13,
wherein the optical splitter includes a beam splitter for separating an amount of light incident through the optical path from the examinee's fundus and an aiming light source spaced apart from the beam splitter with a predetermined distance, disposed at a perpendicular direction to the optical path.

15. The ophthalmic imaging system of claim 14,
wherein the aiming light source includes a main light source and an auxiliary light source spaced apart from the main light source.

16. The ophthalmic imaging system of claim 14,
wherein a wavelength of light emitted from the aiming light source has a visible wavelength.

17. The ophthalmic imaging system of claim 15,
wherein an examinee's viewing angle based on the main light source is symmetric about the optical axis and the examinee's viewing angle based on the auxiliary light source is asymmetric about the optical axis.

18. The ophthalmic imaging system of claim 15,
wherein an optical path of a first light emitted from the main light source is different from that of a second light emitted from the auxiliary light source.

19. An ophthalmic imaging apparatus comprising:
an imaging unit for photographing an examinee's fundus; and
an image generator for generating a fundus image by processing an image of the examinee's fundus photographed by the imaging unit
wherein the imaging unit comprises,
an illumination optical system that illuminates an examinee's fundus with light emitted from a light source;
a photographing optical system that forms an optical path of the light reflected from the examinee's fundus;
an image receiving unit disposed spaced apart from the photographing optical system with a predetermined interval; and
an optical splitter disposed between the photographing optical system and the image receiving unit.
wherein the photographing optical system comprises,
an ophthalmic lens module including a first positive lens and a first converging lens sequentially arranged from the examinee's fundus,
a projection lens module including a negative meniscus with a convex surface facing in an opposite direction of the the examinee's fundus, a second positive lens, a diverging lens, and a second converging lens sequentially arranged from the examinee's fundus, and
an aperture stop disposed on an optical axis of the light reflected from the examinee's fundus between the ophthalmic lens module and the projection lens module.

20. The ophthalmic imaging apparatus of claim 19,
wherein the optical splitter includes a beam splitter for separating an amount of light incident through the optical path from the examinee's fundus and an aiming light source spaced apart from the beam splitter with a predetermined distance, disposed at a perpendicular direction to the optical path.
